(19) United States
(12) Patent Application Publication
(10) Pub. No.: US 2004/0241635 A1
(43) Pub. Date: Dec. 2, 2004

(54) Assay for seed vigour

(76) Inventor: Wayne T. Buckley, Manitoba (CA)

Correspondence Address: Michael R Williams, Ade & Company, 1700 360 Main Street, Winnipeg Manitoba, R3C3Z3, CA

(21) Appl. No.: 10/475,033

(22) PCT Filed: Apr. 18, 2002
(86) PCT No.: PCT/CA02/00538

(30) Foreign Application Priority Data
Apr. 18, 2001 (CA) 2344186
Sep. 20, 2001 (CA) 2357618

(51) Int. Cl. C12Q 1/00

(52) U.S. Cl. 435/4; 47/58.1 R

(57) ABSTRACT

Vigour of the seed of various crops, especially small-seeded crops, is determined by measuring volatile gases emitted from the seed when exposed to various conditions. For example, ethanol produced by canola (Brassica napus and Brassica rapa) seed in an enclosed container stored for 24 hours at room temperature after the seed has been made up to 20% moisture is measured by gas chromatography, other gas monitoring instrumentation or by colour change in an indicator substance. The quantity of ethanol determined by any method of analysis indicates the vigour of the seed.

### FIELD OF THE INVENTION

The present invention relates generally to the field of agriculture. More specifically, the present invention relates to a method for determining seed vigour.

### BACKGROUND OF THE INVENTION

Seed vigour is an important factor in the economical production of field and vegetable crops, and in the quality of malting barley. Field crops, such as canola, may have to be replanted and/or may have lower yield because of low vigour. The seed of vegetable crops is of high value and low-vigour in the seed represents a significant extra cost for seed in addition to production losses. Low-vigour malting barley may loose germination percentage during shipment and storage and no longer be acceptable for malting. Thus, farmers, vegetable growers, seed processors, seed merchants, marketing agents and maltsters need reliable, rapid and economical methods for determining seed vigour.

Gorecki et al. (Gorecki et al., 1985, Can J Botany 63: 1035-1039) examined volatile exudates from germinating pea (Pisum savitum) and showed that as storage time increased, viability decreased. Furthermore, while they observed no qualitative differences in the volatile exudates produced by germinating seeds that had been stored for different periods of time, the quantities of ethanol and acetaldehyde produced by germinating seeds was somewhat proportional to the age of the seed, increasing as seed age increased. However, dry seed produced only small quantities of both volatiles. They also noted that determinations of acetaldehyde and ethanol in the space over germinating seeds by means of gas chromatography might be a useful seed vigour test. Gorecki et al. (Gorecki et al., 1992, Acta Physiologiae Plantarum 14: 19-27) also analyzed volatile organics produced by starchy pea seeds and fatty cocklebur (Xanthium pennsylvanicum) seeds following water imbibition, wherein aged seeds showed increasing amounts of ethanol and acetaldehyde produced, although in cocklebur seeds, acetaldehyde was the preponderant end product. As will be apparent to one knowledgeable in the art, imbibition is the movement of water into the seed, which in turn causes seed swelling and is essential for germination of the seed.

Muskmelon (cantaloupe, Cucumis melo) seeds were aged artificially for up to 12 d at 45° C. and 100% relative humidity. Artificial ageing reduced their ability to germinate and increased the production of ethanol and acetaldehyde during imbibition. The authors suggested that ethanol production in the first hours of imbibition might be used as a method to predict germination in muskmelon seed (Pesis and Ng, 1984, J. Expt. Bot. 35: 356-365.).

Zhang et al. (Zhang et al., 1994, Seed Science Research 4: 49-56) hypothesized that seed-evolved volatiles cause the loss of seed germinability during storage. It is of note that the seeds were stored at 70% relative humidity and that the moisture content of the seeds was approximately 6-14%. Various aldehydes were supplied to lettuce, soybean, sunflower carrot and rice seeds during storage, some of which showed toxicity to seed germinability. Based on these facts, they suggested that endogenous volatiles, especially aldehyde, might be an important factor that accelerates seed deterioration, which often occurs under lower relative humidity and/or temperatures throughout long-term storage. This in turn suggests that removal of acetaldehyde and ethanol from seed storage containers would improve seed vigour, or at least prevent further deterioration, and that it is the amount of ethanol or acetaldehyde that a seed is exposed to that determines vigour.

Naturally or artificially aged soybean (Glycine max) seed had higher ethanol and acetaldehyde concentrations in seed tissue than did un-aged seed; however, the difference between aged and un-aged seed varied greatly with hours of imbibition, and rate of water uptake and temperature during imbibition (Woodstock and Taylorson, 1981, Plant Physiol. 67: 424-428).

The quantity of certain volatile aldehydes (butanal, hexanal and pentanal) when released by heat from dry soybeans and expressed as a percentage of the total volatiles collected were correlated to seed germination and seed vigour (Hailsones and Smith, 1989, Seed Sci & Technol. 17: 649-658).

Unspecified, volatile aldehydes released from germinating soybean seeds were correlated with field emergence of various seed lots in the field. The authors suggested that the determination of aldehydes during germination might be a useful vigour test (Wilson and McDonald, 1986, Seed Sci. & Technol. 14: 259-268).

Decline in germination and field emergence of bean (Phaseolus vulgaris) seed previously subjected to accelerated aging at 42° C. and 100% relative humidity was correlated with a decline in ethylene production by the seed during imbibition (Samimy and Taylor, 1983, J. Amer. Soc. Hort. Sci. 108: 767-769.).

As discussed above, seed vigour is an important factor in the economical production of crops. The ability to rapidly and reliably determine seed vigour would significantly reduce costs associated with yield loss, replanting and return of seeds. There are numerous procedures for determining seed vigour in the art, however, more accurate and rapid procedures are desirable.

### SUMMARY OF THE INVENTION

According to a first aspect of the invention, there is provided a method of measuring seed vigour comprising:

placing a seed under conditions wherein seed metabolism initiates but the seed is not germinating; and measuring the quantity of at least one gas produced by the seed.

According to a second aspect of the invention, there is provided a device for assaying seed vigour comprising:

a quantity of a detector compound that changes colour when exposed to ethanol;

an air-tight container including a sealable opening for placing seeds to be assayed within the interior of the container; and

a regulator for controlling the rate of diffusion of the ethanol from the interior of the container to the detector compound.

According to a third aspect of the invention, there is provided a kit for assaying seed vigour comprising:

a quantity of a detector compound that changes colour when exposed to ethanol;

an air-tight container including a sealable opening for placing seeds to be assayed within the interior of the container; and

a regulator for controlling the rate of diffusion of the ethanol from the interior of the container to the detector compound.

### BRIEF DESCRIPTION OF THE DRAWINGS

FIG. 1 shows chromatograms from headspace gas analysis of high-vigour (top) and low-vigour (bottom) canola seed. Both chromatograms are drawn to the same scale. Note that the ethanol peak on the lower chromatogram has been truncated.

FIG. 2 shows the relationship between amounts of ethanol (expressed as gas chromatography peak areas) in head space gas and bioassay results for 26 canola seed samples (sample set 1). Thirteen pairs, each containing a high- and a low-vigour sample of one of a number of varieties were examined. The low-vigour reference biomass was expressed as a percentage of the high-vigour reference biomass in each pair in order to correct for genetic variation in growth rate from variety to variety. Genetic variation would otherwise have exacerbated measured differences in seedling vigour in the reference bioassay. The shaded region incorporates peak areas less than 500,000 counts and indicates an ethanol range that may be associated with high vigour.

FIG. 3 shows the relationship between amounts of acetaldehyde (expressed as gas chromatography peak areas) in head space gas and bioassay results for 26 canola seed samples (sample set 1). The samples were paired as described in FIG. 2.

FIG. 4 shows the relationship between amounts of unknown E (expressed as gas chromatography peak areas) in head space gas and bioassay results for 26 canola seed samples (sample set 1). The samples were paired as described in FIG. 2.

FIG. 5 shows the relationship between amounts of ethanol (expressed as gas chromatography peak areas) in head space gas and bioassay results for 93 canola seed samples, which include 32 varieties or hybrids and 19 seed treatment formulations of fungicide and insecticide (sample set 2). The shaded region incorporates peak areas less than 500,000 counts and indicates an ethanol range that may be associated with high vigour.

FIG. 6 shows the effects of seed moisture percentage on amounts of ethanol (expressed as gas chromatography peak areas) in head space gas of high- and low-vigour canola seed lots. The head space gas was analysed 24±2.5 h after water was added to the seed.

FIG. 7 shows a device for colourimetric determination of seed vigour. Seed of high and low vigour was added to 250-ml flasks, seeds were brought up to 20% moisture and the flasks were sealed and fitted with ethanol-indicating diffusion tubes. The figure shows the colour development after 24 hours at room temperature.

FIG. 8 is a close-up of FIG. 7.

FIG. 9 is a side view of containers of canola seed. These containers have an integrated colour disc in the lid for colourimetric determination of vigour.

FIG. 10 shows a device for colourimetric determination of canola seed vigour by means of an integrated colour disc in the container lid. FIG. 10 is a top view of the containers shown in FIG. 9. High-vigour (left) and low vigour (right) canola seed was made up to 20% moisture and sealed in the containers for 24 h.

FIG. 11 shows a device for colourimetric determination of canola seed vigour by means of an integrated colour disc in the container lid. High-vigour (left) and low vigour (right) canola seed was made up to 20% moisture and sealed in the containers for 24 h.

FIG. 12 shows the determination of gaseous ethanol standards in the concentration range suitable for estimation of seed vigour by analysis of head space gas. A commercial hand-held gas monitoring instrument was used for the determinations.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

### Definitions

As used herein, “seed vigour” refers, depending on the context, to the ability of seed to germinate rapidly and achieve a high percentage of germination; to produce seedlings that emerge rapidly from the soil and have a high percentage of emergence; to produce seedlings that grow rapidly and demonstrate superior tolerance to various stresses including but not limited to cold, weeds and insects; and to the ability of seed to withstand storage or shipment with a minimum loss of the ability of seed and its seedlings to germinate, emerge from the soil, grow and tolerate stresses. Not all aspects of poor vigour may be found in the same seed. For example, low-vigour seed may have a high germination percentage but produce seedlings that grow slowly compared to high-vigour seed of the same genotype grown under the same conditions.

As used herein, “moisture” refers to the amount of water present in a material expressed as a percentage of the undried weight of the material.

As used herein, “head space” refers to the space over a substance in a sealed container. As used herein, head space gas refers to the gases in the head space.

As used herein, “hypocotyl” refers to the section of stem of a seedling between the cotyledons and the root-shoot junction.

As used herein, “small”, “medium” and “large” seed refers to seeds wherein there are more than 100 seeds per gram (small, s), more than 10 and up to 100 seeds per gram (medium, m) and 10 or fewer seeds per gram (large, I).

Described herein is a method of measuring vigour of various types of seed by measuring volatile gases, for example, ethanol, emitted from the seed when exposed to various conditions. These conditions may include, for example, exposing the seed to sufficient moisture to initiate metabolism but less moisture than required for germination, purging the air in a sealed container with nitrogen or other gases, and including various inhibitors or stimulators of metabolism in the water added to the seed. For example, ethanol produced by canola (Brassica napus and Brassica rapa) or barley (Hordeum vulgare) seed in an enclosed container stored for 24 hours at room temperature after the seed has been made up to 20% moisture is measured by gas chromatography, other gas detection instrumentation or by colour change in an indicator substance. For illustrative purposes, an example of other gas detection instrumentation is the Pac III Single Gas Monitor manufactured by Dr≅ger, Sicherheitstechnik GmbH, Luebeck, Germany. As discussed below, the quantity of ethanol determined by any method of analysis indicates the vigour of the seed.

Plants and other organisms obtain energy from stored carbohydrate by either aerobic or anaerobic metabolism. Aerobic metabolism converts carbohydrate to carbon dioxide and water, whereas anaerobic metabolism converts carbohydrate to ethanol, other organic compounds and carbon dioxide by fermentation. The former is more energetically efficient than the latter. As described below, when seed ages or looses vigour for other reasons, there appears to be a shift to a higher proportion of anaerobic metabolism with elevated ethanol production compared to more healthy seed of the same species.

As will be appreciated by one knowledgeable in the art, 20% moisture is sufficient to initiate metabolism within the seed but is not sufficient moisture to induce germination. It is of note that other suitable moisture levels and incubation times which induce similar conditions as well as other methods which induce these conditions are within the scope of the invention and may be used within the invention. For example, seed moisture may be 10-50%, 15-40% or 15-35%. The incubation time may vary from 2 hours to 48 hours or from 18 to 30 hours.

As will be appreciated by one knowledgeable in the art, other suitable means known in the art may also be used to determine the amount of ethanol or other gases emitted from the seeds. See for example, U.S. Pat. No. 3,455,654, U.S. Pat. No. 3,223,488, U.S. Pat. No. 3,208,827, or U.S. Pat. No. 2,939,768.

In one embodiment of the invention, there is provided an on-farm assay device as well as a kit containing the components thereof, as discussed below. The assay device comprises: a quantity of a detector compound that changes colour when exposed to ethanol; an air-tight container including a sealable opening for placing seeds to be assayed within the interior of the container; and a regulator for controlling the rate of diffusion of the ethanol from the interior of the container to the detector compound. It is also of note that the device is arranged such that the detector compound may be viewed while it is exposed to the head space gas from the seed.

Examples of suitable detector compounds are described herein and are also well known in the art. As will be apparent to one of skill in the art, the exact concentration of detector compound used may vary according to the specific seed and the assay conditions.

In some embodiments, the regulator is a perforated piece of Teflon with the number and size of holes carefully controlled. As will be apparent to one of skill in the art, the rate of diffusion of ethanol is controlled by the regulator and depends in some embodiments on the size and number of the holes. The rate of diffusion will determine the rate of colour change of the detector compound. This, along with the quantity of seed and size of container will permit colour change at the desired seed vigour threshold (probably around 80%) over a specified time period (approximately 24 h). As will be appreciated by one of skill in the art, other suitable times and percentages may also be used.

In some embodiments, an inert support, for example, a glass fiber disc, is impregnated with the detector compound. As will be apparent to one of skill in the art, other suitable supports include, but are by no means limited to unreactive porous materials (e.g. a porous ceramic disc) or powders (e.g., diatomaceous earth).

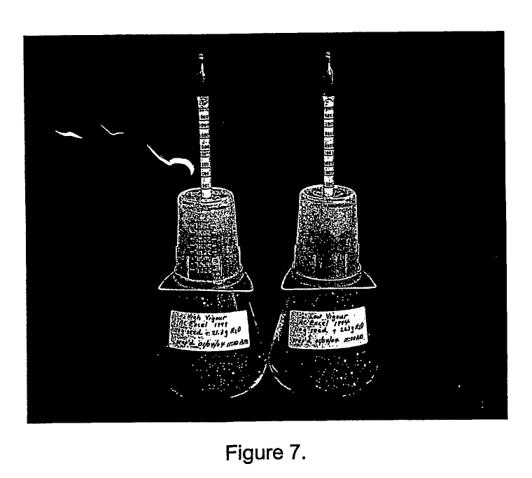

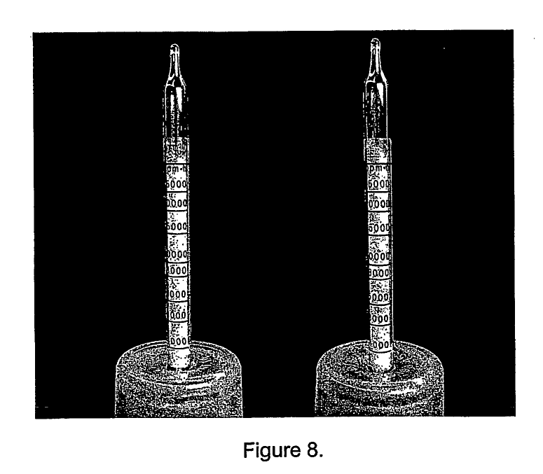

According to another embodiment of the invention, there is provided a device for measuring seedling vigour comprising a substantially airtight container and an ethanol detector as described above. In use, the moisture content of the seed to be tested is measured and is elevated to 10-50%, 15-45% or 15-35% as discussed below, if necessary, and the seed is placed in the air tight container. The ethanol detector is then operably connected to the airtight container such that ethanol evolved from the seed is measured by the ethanol detector. Evolved ethanol may be measured from 2 hours up to 48 hours or longer, as discussed above. An example of such a device is shown in FIGS. 7 and 8, wherein the substantially airtight container is a sealed 250-ml flask and the ethanol detector is an ethanol-indicating diffusion tube. As will be appreciated by one knowledgeable in the art, other suitable containers and detectors may also be used. For example, a colourimetric ethanol detector may be constructed as an integral part of the container. Also for example, the headspace gas may be analysed directly or indirectly by gas chromatography or other instrumental procedures. It is of note that there are 300-500 canola seeds in a gram. One-half to 4 g of seed in 2-ml to 12-ml vials are used in the routine gas chromatography procedure. Versions of the colourimetric on-farm assay use 5 to 250 g. Similar quantities may be used for other seeds.

The invention provides kits for carrying out the methods of the invention. Accordingly, a variety of kits are provided. The kits of the invention comprise one or more containers comprising an air-tight container, a quantity of detector compound, at least one regulator and a set of instructions, generally written instructions although electronic storage media (e.g., magnetic diskette or optical disk) containing instructions are also acceptable, relating to the use of the kit for the intended purpose. As discussed above, an inert carrier may be impregnated with detector compound and may be arranged to be mounted onto the container or connected to the container for communication therebetween. The kit may include a plurality of carriers impregnated with detector compound. In some embodiments, the plurality of carriers may be impregnated with different detector compounds or mixtures of different compounds and/or may be impregnated with different levels of the compounds. In addition, a plurality of regulators may be provided. In some embodiments, the respective regulators may be arranged to restrict gas flow to differing extents. In yet other embodiments, the kit may include a high osmotic strength water solution for adding moisture to seeds.

It is further of note that in some embodiments, the kit components are impervious to water vapour and do not give off organic vapours. This is important in achieving an acceptable shelf life for the kit. The kit will discolour slowly by itself unless water vapour and organic vapours can be eliminated. In these embodiments, the container may be composed of glass and/or Teflon.

The invention will be described by way of examples. However, the invention is not limited to the examples. In the examples, the seed of a number of crops are analysed. However, other suitable seeds, for example, other grains, oilseeds, vegetables, horticultural crops, fiber crops, speciality crops, pharmaceutical and nutriceutical crops and non-food crops may also be analysed. Examples of seeds suitable for analysis with the invention include but are by no means limited to alfalfa, barley, buckwheat, cabbage, canola, clover, flax, lentils, mustard, sunflower, turnip and wheat.

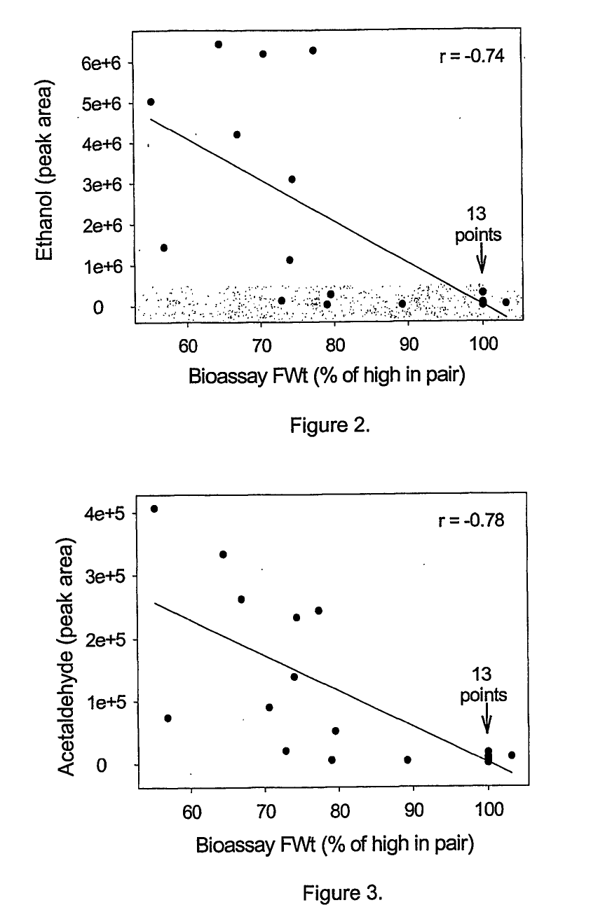

As can be seen in FIGS. 2 and 5, a gas chromatography peak area for ethanol of greater than 500,000 appears to be associated with low vigour in canola. It is of note that gas chromatography techniques (including head space gas analysis and solid phase micro extraction) may yield different results depending on details of the technique. This could be overcome by converting peak areas into gaseous concentrations.

The most important volatile is ethanol, followed by acetaldehyde and unknown E.

As discussed in Example VI, the correlation coefficients found at two moisture levels show that the accuracy of the vigour test is lower at 35% compared to 20% moisture. Some insight into what this means may be obtained by performing some crude calculations on the accuracy, which is described in Example III. In this set of 26 samples, the vigour of I sample was incorrectly diagnosed. This calculation is based on a 500,000 threshold and specifying rejection of seed that has less than 79% of the vigour expected for high quality seed of the same variety. (A cut off of about 80% has been suggested by representatives of the canola industry.)

Measurement of air-dried seed moisture can be accurately done in the laboratory. Once the moisture percentage is known, the appropriate amount of water can be added to achieve the desired final moisture percentage. Most farmers have a moisture meter or ready access to moisture determination. In some embodiments, the user may determine the moisture content, then add the appropriate amount of water in order to achieve the required moisture percentage. Alternatively, a water solution of high osmotic strength (such as a polyethylene glycol solution) could be used to limit the moisture uptake of the seed to a certain percentage.

While not wishing to be bound to a specific theory, it appears as though most crops produce high amounts of ethanol when vigour is low. Only some crops, though, like canola, have low ethanol emissions when vigour is high. Those that have relatively high ethanol emissions from high-vigour seed may include the three main crops discussed in the relevant literature, peas, beans and soybeans. The difference in ethanol between low and high vigour with these crops may not be great enough to develop a test.

### EXAMPLE I

### Determination of Reference Vigour Status

A biological assay was used to determine the reference, or “true”, vigour status of various seed lots. The reference vigour status of each sample of seed was determined by germinating about 100 seeds on a stainless steel screen suspended about 1 cm above an aerated complete nutrient solution. Aerosol from the bubbling solution was sufficient to thoroughly wet the screen and seed, thus providing conditions for germination. Reference vigour determinations were performed in chambers with the following day and night conditions: day—16 hours light (55-60 μmol s⁻¹ m⁻²), >90% RH, 22° C.; night—8 hours dark, >90% RH, 17° C. Two variations of the biological reference assay were employed. In the first variation, percentage germination and percentage of germinated seedlings with greater than 7 mm hypocotyl elongation were measured each day for five days after the start of imbibition. At the end of the fifth day the seedlings were harvested and fresh and dry weights of roots and shoots determined. A vigour rating scale was established based on the range of observed values for germination, hypocotyl elongation and root and shoot biomass production. In the second variation, daily measurements of germination and hypocotyl elongation were not performed, although seedlings were harvested and weighed at the end of the fifth day as described. Vigour rating was based on fresh weight of roots and shoots. Although the first variation was more elaborate and provided more information concerning physiology and growth, both variations provided similar vigour assessments.

### EXAMPLE II

### Gas Chromatography Method of Determining Vigour

Seed was weighed into vials of various sizes. If required, water was added to the seed to make it up to desired moisture contents. The vials were sealed and incubated for 24 h at selected temperatures. Subsamples of seed were dried previously to constant weight at 60° C. in order to determine the moisture content of the original seed sample. After 24 hours, volatile compounds that had accumulated in the headspace gas of the seed were analysed by gas chromatography. Analytes were pre-concentrated and separated from water vapour in the headspace gas by means of automated solid phase micro-extraction. A 30-meter capillary column with a non-polar immobile phase was used to separate the analytes, which were detected and quantified by means of a flame ionization detector.

The separated analytes appeared as peaks in the gas chromatography output. The peaks were identified by two procedures. Firstly, purified known compounds were introduced into the seed head space gas and empty vials in order to match the retention time of known compounds with those of unknown peaks in the chromatograms. Secondly, the gaseous output from the gas chromatography column was introduced into a mass spectrometer and information on the masses of eluted compounds and their degradation products was obtained. Compounds were identified by correlation of the measured masses with those of known compounds. Mass spectrometric analysis was performed by Dr. G. Eigendorf, University of British Columbia.

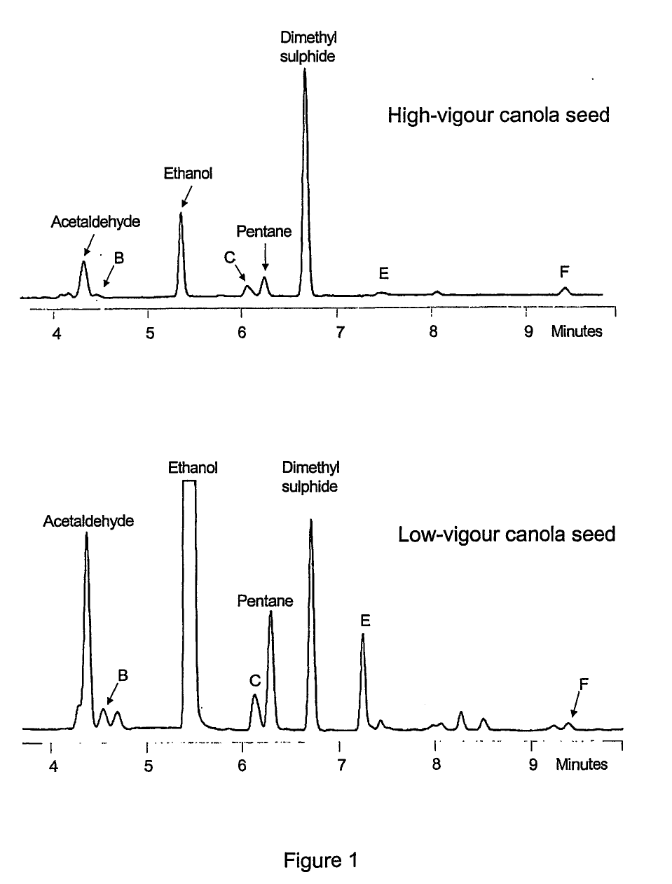

Typical chromatograms obtained from gas chromatographic analysis of the headspace gas over high- and low-vigour canola seed lots are shown in FIG. 1. The ethanol peak appearing at approximately 5.5 min was much larger in most of the low-vigour seed samples compared to the high-vigour seed samples.

Ethanol, acetaldehyde and Unknown E were found to be highly correlated with vigour (correlation coefficients >0.7). Acetaldehyde is an intermediate found in the conversion of glucose to ethanol. It is of note that Unknown E may represent another volatile intermediate or endproduct in the metabolism of glucose, such as pyruvic acid or acetic acid. On the other hand, Unknown E may be unrelated to the metabolism of glucose and may be any low-molecular weight, volatile compound generated by plants. Furthermore, it has been shown that pentane is present in the head space gas and is weakly correlated (correlation coefficient <0.5) to vigour. Pentane is an end product of fatty acid oxidation and may play a role in diagnosing seed quality as well. Other compounds found to be weakly correlated with vigour were Unknown B, Unknown C and Unknown F. Preliminary data indicates that Unknown B may be propane. It is possible that a vigour rating determined as a combination or combinations of these peaks may be more reliable than a vigour rating using ethanol alone. The potential combinations may be obtained by calculated functions of measured quantities of individual compounds or by analytical techniques that measure one or more of the compounds as a group and may not distinguish among the compounds. As will be appreciated by one knowledgeable in the art, the gases may be measured using means and/or indicator compounds known in the art. One compound, dimethyl sulphide, was not significantly correlated to vigour.

### EXAMPLE III

### Evaluation of Gas Chromatography Method of Determining Vigour

Samples of 13 pairs of canola seed lots (Brassica napus) were obtained. Each pair consisted of a sample of a high-vigour and a low-vigour seed lot. In addition, both members of each pair were the same variety of canola. The original designations of high and low vigour for these seed lots were estimates only.

All 26 seed samples were subjected to the reference vigour bioassay as described above. It was found that seed samples of different varieties of canola differed somewhat in their vigour, apparently due to genetic variation. Since all varieties of canola must have adequate yield before they can be registered, we are not concerned with measuring genetic variations in vigour among varieties, but, instead, we wish to identify those seed lots that have less than optimal vigour within their variety. Thus, in order to compare the low-vigour seed lots to the high-vigour lots, the high-vigour seed lots were assigned a vigour status of 100. Within each pair, the low-vigour lot was compared to the high-vigour lot with respect to shoot fresh weight, root dry weight, germination rate and hypocotyl elongation rate. Each parameter for a low-vigour lot was expressed as a percentage of the parameter value for its paired high-vigour lot. The mean of the four percentage values was taken as the vigour rating for each low-vigour seed lot.

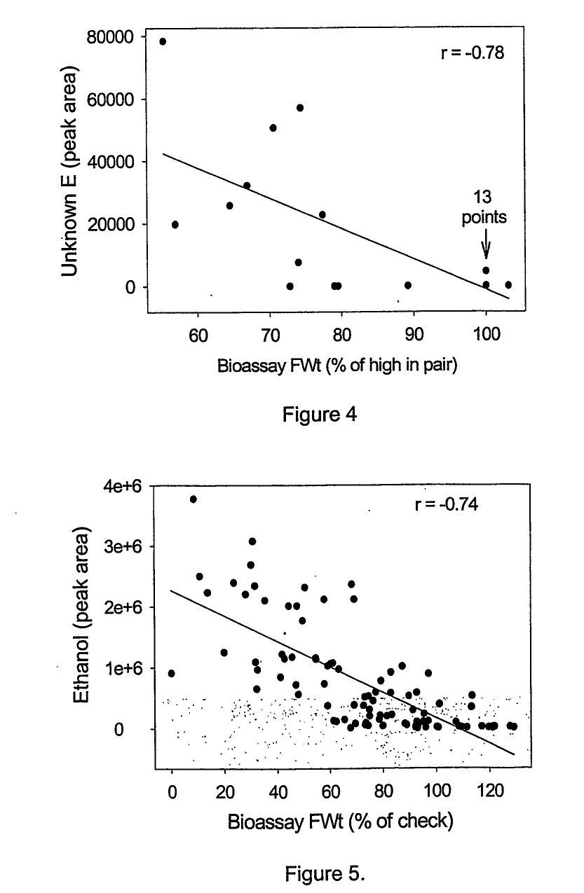

Analysis of volatiles in headspace gas of each seed sample (after storage at 20% moisture for 24 hours at room temperature) was determined in triplicate by gas chromatography. Significant correlations (P<0.05) between the reference vigour rating and measurements of ethanol, acetaldehyde and unknown E in the headspace gas are shown in FIGS. 2-4. The ethanol found in headspace gas of each canola seed lot is compared with its reference vigour value in Table 1.

Because sample set 1 is composed of paired high- and low-vigour seed lots of the same varieties, it is suitable for estimating the accuracy of the head space gas method of determining vigour. Representatives of the canola industry have indicated that a vigour test should be capable of identifying seed samples that have lost more than about 20% of their vigour (we will choose 21% for the current accuracy estimate). In addition, results of ethanol analysis in this and other sample sets shows that ethanol peak areas up to 500,000 are consistent with high vigour. Thus, vigour will be incorrectly diagnosed if 1) reference vigour is less than 79% and ethanol peak area is less than 500,000, or 2) reference vigour is 79% or greater and ethanol peak area is 500,000 or greater. Based on these criteria only one of the 26 samples in sample set 1 would be incorrectly diagnosed.

### EXAMPLE IV

### Evaluation of the Gas Chromatography Method by Analysis of Canola Seed of Different Varieties and Pesticide Treatments

Ninety-three samples of canola seed (Brassica napus and Brassica rapa) were collected from farmers, seed laboratories, seed merchants and other sources. The collection (sample set 2) included 32 varieties or hybrids of canola that were either untreated or treated with 19 different formulations of fungicide and insecticide. The set of 93 samples represented canola genetics and seed treatments in common use in agriculture. About half of the seed samples were found to be low vigour as determined by the reference bioassay. The samples, though, are too diverse with respect to genetics and seed treatments to be organized in high/low vigour pairs as was done in sample set 1. Duplicate 0.5-g subsamples of seed were made up to 20% moisture in sealed vials and, after 24±2.5 h at room temperature, were subjected to head space gas analysis by gas chromatography. Results for ethanol determinations are shown in FIG. 5. Because the data cannot be presented in high/low vigour pairs, variation in the horizontal axis includes genetic variation as well as vigour loss. We cannot distinguish between these two sources of variation in unpaired samples. Correlations of all measured low molecular weight components with results of the reference vigour bioassay are shown in Table 2. The results of sample set 2 show that ethanol, acetaldehyde and Unknown E are good indicators of seed vigour in a diverse collection of canola seed samples.

### EXAMPLE V

### Relationship Between Gas Chromatography Results, Reference Bioassay Results and Other Measures of Vigour

Seventeen samples of untreated canola seed lots (sample set 3) were subjected to head space gas analysis, reference bioassay and a series of tests used in the art as measures of vigour for a variety of crops. Although there are many tests of vigour used in the art, field biomass, cold stress and germination tests were selected for study. Recent research (R. Elliott. 2002. Presentation to the Seed Vigour Research Review, Canola Council of Canada, Saskatoon.) indicated that they are the most useful of available tests for estimating canola vigour. It is generally agreed in the art that seedling biomass determinations in the field are the most accurate representation of vigour. However, field measurements are not normally performed because they are laborious, costly and usually cannot be performed before spring seeding. Several laboratory-based cold stress tests are used to estimate the ability of seed lots to tolerate cold soil. The most common test of vigour is the standard 7-d germination test. Germination tests of shorter duration may more accurately estimate vigour because they tend to estimate rate of germination rather than total germination (W. Buckley, unpublished data). Dr. R. H. Elliott, Agriculture and Agri-Food Canada, kindly provided data on the analysis of sample set 3 by the field biomass, cold stress and germination tests. Correlations of field biomass, cold stress and germination test results with the reference bioassay and ethanol results are shown in Table 3. Both the reference bioassay results and the ethanol results were strongly correlated with the other vigour measurements. The results provide additional evidence that determination of ethanol in head space gas of moist seed is a reliable method of determining vigour.

### EXAMPLE VI

### Effect of Seed Moisture Percentage on Determination of Ethanol in Head Pace Gas

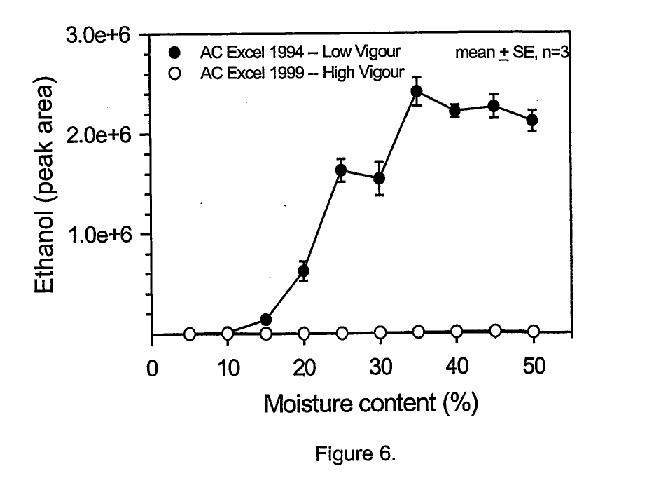

One-gram samples of high- and low-vigour canola seed were weighed into 10-ml head space gas sampling vials. Water was added to adjust the seed moisture from 5 to 50% and low-molecular weight volatile compounds in the head space gas were determined after 24±2.5 h incubation at room temperature. Moisture percentage had a pronounced effect on the evolution of ethanol from low-vigour canola seed and only a minor effect on the high-vigour seed (FIG. 6). Similar results were obtained for acetaldehyde and Unknown E. From these results it might appear that a moisture content of about 35% might be ideal for the head space gas analysis. However, variation in analytical results increased with higher moisture percentages. To test the significance of this effect on the ability to distinguish between high- and low-vigour samples, a set of canola seed lots was tested at 20 and 35% moisture (seed set 4; 40 samples of several varieties either treated or untreated with fungicide; approximately equal numbers of high- and low-vigour seed lots; note that seed set 3 was a subset of seed set 4). Correlation coefficients between the reference vigour test and ethanol, acetaldehyde and Unknown E were −0.78, −0.72 and −0.73, respectively, for the seed at 20% moisture, which is consistent with the other sample sets studied. However, at 35% moisture correlation coefficients were −40.60, −0.57 and −0.62, respectively. The results indicated that the ability to resolve high- and low-vigour seed based on ethanol, acetaldehyde or Unknown E determinations is substantially better at 20% moisture compared to 35% moisture. Thus, control of the moisture percentage of the seed is important when using ethanol determinations to estimate seed vigour. Adding water to seed without controlling the moisture percentage within reasonable limits may lead to unreliable determinations. The optimum moisture percentage may be in the range of 15 to 25%. Seed germination begins at 35 −40% moisture in canola seed.

### EXAMPLE VII

### Estimating Germination Loss in Malting Barley

The results of our work suggest that loss of germination is one of a series of changes that happen to seed during loss of vigour. Loss of germination, though, occurs relatively late in the deterioration process, whereas the appearance of ethanol in headspace gas occurs relatively early. Thus, seed ethanol emissions are expected to be a good predictor of germination loss. In the malting process, barley is germinated, then dried to yield malt. A high germination percentage is very important. After barley has been selected for malting, though, it is usually stored or transported for varying lengths of time. By the time it enters the malting process, the barley may have lost significant germination percentage. It would be advantageous to identify those barley lots with a predisposition for germination loss as early as possible.

Thirty barley seed lots were tested for ethanol accumulation in head space gas. Four grams of seed was made up to 20% moisture and sealed in 10-ml head space gas vials and incubated for 24±2.5 h at room temperature prior to gas chromatography analysis. The seed was also tested for susceptibility to germination loss by artificial aging at elevated temperature and moisture (the germination loss data was kindly provided by Dr. M. Izydorezyk, Canadian Grain Commission). Results of the analyses are shown in Table 4. The data indicate that elevated ethanol in head space gas may be a suitable predictor of germination loss in barley.

### EXAMPLE VIII

### Suitability of the Head Space Gas Analysis Method of Determining Seed Vigour in a Variety of Crops

The suitability of the head space gas analysis method for determining vigour in a variety of crops was examined by means of a screening procedure. The procedure involved the determination of ethanol in head space gas of un-aged and artificially aged paired samples of seed. Seed samples of a number of crops were obtained from various sources. Duplicate 4-g subsamples were weighed into 10-ml head space gas analysis vials. Sufficient water was added to make the seed up to 20% moisture (based on an assumed air-dried moisture content of 3%) and the vials were sealed. One subsample was maintained at room temperature while the other was elevated to 40° C. for 24 h then cooled to room temperature. Gas chromatography analysis of head space gas was performed 24-29 h after the addition of water. Single or double analysis of each sample was performed.

Accelerated, or artificial, aging of seed is a technique commonly used in the art for the determination of seed vigour. Seed is maintained in a humid environment, or at an elevated moisture percentage, and at an elevated temperature for periods of time that may vary from hours to weeks. Usually the environment is 100% relative humidity and the temperature is in the range of 30-40° C. After accelerated aging has been performed, the germination percentage of aged seed is determined. Seed that shows a higher germination after accelerated aging is considered to have higher vigour. It can be appreciated that the current screening procedure is a combination of accelerated aging and head space gas analysis. It is reasoned that an increase in ethanol emissions by seed subjected to accelerated aging indicates that the seed is likely to undergo similar changes as vigour declines during normal storage. Thus, seed lots that show a high ratio (greater than 10) of aged to un-aged ethanol emissions are considered to be good candidates for vigour determination by head space gas analysis. The percentage error in diagnosing vigour from head space gas analysis for crops with low ratios may be unacceptable.

Thirty crops were tested with the screening procedure (Table 5). All crops examined showed an increase in ethanol emissions, most to relatively high amounts, following artificial aging. This suggests that elevated ethanol emission may be a universal phenomenon in seed that has undergone deterioration or vigour loss. The ratio of ethanol emissions from aged and un-aged seed varied from 472 in canola to 2 in vetch and barley. High ratios were associated with lower ethanol emissions from un-aged seed and not higher than average emissions from aged seed.

The results for barley (Table 5) were not expected. Although five lots of malting barley, including four varieties, were tested no sample emitted low ethanol before aging. However, it is apparent from the results in Table 4 that high quality barley does have low ethanol emissions. There may be several reasons for the unexpected barley results. 1) Conditions have been poor for the past three years in Western Canada for the production of malting barley. 2) Barley, especially in the eastern prairies tends to be infected with fusarium, which reduces quality. 3) Malting barley must be stored under carefully controlled conditions in order to maintain its quality after harvesting. Two of the five samples screened were not properly stored and storage conditions of the other three are unknown.

As described in the Background to the Invention, there were four references found in the scientific literature that referred to the possibility of measuring seed vigour by analysis of gasses emitted from imbibing or germinating seed. Three of these referred to ethanol or acetaldehyde and one referred to ethylene. There were four crops in these studies: muskmelon, pea, soybean and snapbean (a type of bean); and one weed: cocklebur. The four crops are represented in Table 5. Muskmelon yielded a ratio of 109, the fourth highest value in the survey. However, muskmelon emitted the lowest amount of ethanol from aged seeds of any of the crops studied. Although this would not be a limitation for analysis by gas chromatography, analysis by the colourimetric method or the instrumental method (Pac III) may not be feasible because of low ethanol. Peas, soybeans and beans were found to have aged:un-aged ethanol ratios less than 10. Although we cannot discount the possibility that we were unable to find a single high quality sample of seed for these crops, it appears most likely that high-vigour seed of these crops normally emits relatively high amounts of ethanol. The differences in ethanol and acetaldehyde emissions between high- and low-vigour seed samples of these crops probably are not great enough to develop a reliable assay. This might explain why a vigour assay based on gaseous emissions from seed has not been developed previously.

Small-seeded crops appear to be more suited for the ethanol vigour assay than large-seeded crops. Six of the seven highest ratios were from small-seeded crops (Table 5). The tendency for medium and large-seeded crops to emit greater amounts of ethanol from un-aged seed may be related to the physical size of the seed as well as the quantity of carbohydrate reserves available in the seed. Large seeds have larger carbohydrate reserves per embryo and tend to have a larger percentage of carbohydrate by weight. Because carbohydrate may be in relatively short supply in small seed, healthy small seed may have a greater tendency to avoid fermentation than healthy larger seed, thereby conserving the energy supply. Fermentation, which produces ethanol from carbohydrate, is energetically inefficient, and normally occurs when oxygen is limiting. Oxygen may be limiting in the sealed vials of the ethanol assay. More importantly, though, the interior of a large seed may have limited oxygen because of the distance required for diffusion of oxygen from the surface. Thus, un-aged large seeds might be expected to emit more ethanol than un-aged small seeds.

### EXAMPLE IX

### Colourimetric Method of Determining Vigour Using Commercial Diffusion Tubes

Canola seed was weighed into containers of various sizes. Water was added to the seed to make it up to 20% moisture. The containers were sealed and an ethanol indicator tube (Dräger Diffusion Tube, Ethanol 1000/a-D, Dräger Sicherheitstechnik GmbH, Germany) was inserted into a tight-fitting hole in the vial lid. The vials were incubated for 24 h at room temperature. Sub-samples of seed were dried previously to constant weight at 60° C. in order to determine the moisture content of the original seed sample. After 24 hours, the extent of colour change in the tube indicated the quantity of ethanol emitted by the seed and, therefore, the vigour of the seed. The quantity of ethanol given off over 24 hours by low-vigour canola seed was sufficient to cause a colour change in the commercial ethanol indicator tube. Thus, the feasibility of a simple, economical on-farm test based on colour change was demonstrated. An example of the difference in colour development 24 hours after moistening high- and low-vigour seed is shown in FIGS. 7 & 8.

### EXAMPLE X

### Colourimetric Method of Determining Vigour Using an Integrated Colour Indicator

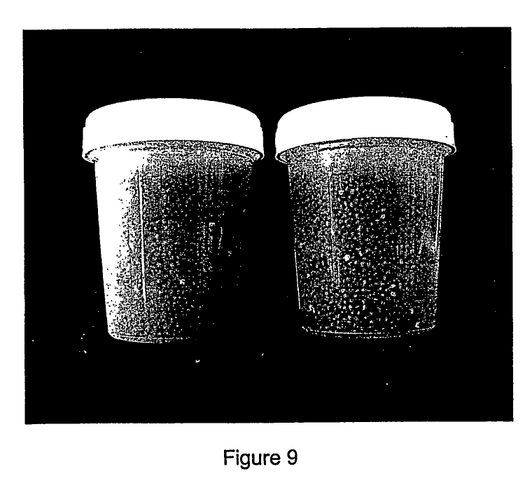

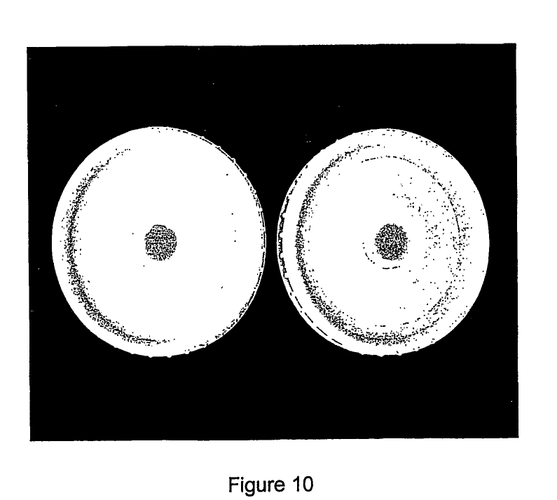

The quantity of ethanol and related metabolites given off over 24 hours by low-vigour canola seed at 20% moisture caused a colour change in a colour indicating disc that was an integral part of the seed container. The colour disc consisted of 80 microliters of a colour developing reagent absorbed into a 10-mm diameter glass fiber disc. The reagent consisted of 10 ml sulphuric acid and 1.2 g potassium dichromate per 100 ml of water solution. The glass fiber disc with reagent was dried over a desiccant prior to assembly of the seed container. The container was constructed so that the colour disc was exposed to gasses emitted from the seed while the disc was visible through a clear plastic window. An example in which the containers are 4-ounce hospital-type specimen cups containing 60 g of seed made up to 20% moisture 24 h prior to recording the colour development is shown in FIGS. 9 and 10. EXAMPLE XI

### Colourimetric Method of Determining Vigour Using an Integrated Colour Indicator

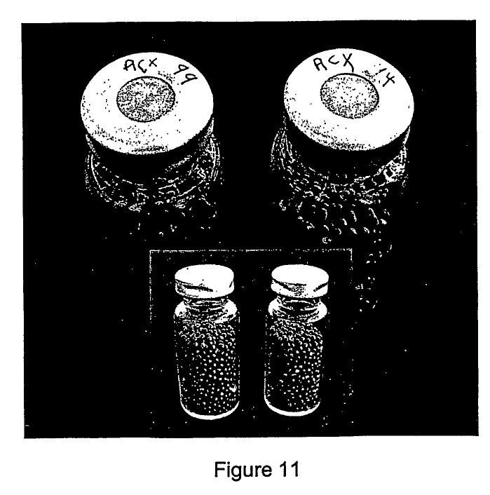

Five grams of canola seed was added to 10-ml glass vials. The seed was made up to 20% moisture and the vials sealed with crimp tops that included an integrated colour disc. The colour disc was the same as described in Example X. FIG. 11 shows the colour development obtained with high- and low-vigour seed 24 hours after adding water to the seed.

### EXAMPLE XII

### An Instrumental Method of estimating Seed Vigour by Determining Volatiles Emitted by Moist Seed.

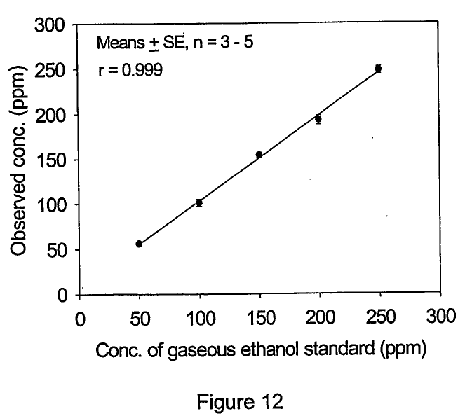

Commercially available instrumentation other than gas chromatography may be suitable for measurement of seed vigour. The instrumentation may have the advantages of lower cost and require less training than gas chromatography. FIG. 12 shows the determination of gaseous ethanol at concentrations suitable for the analysis of head space gas of moist seed using a hand held gas monitoring instrument (model Pac III; Dreiger).

### EXAMPLE XIII

### Summary

A method for the determination of vigour in seed has been described, and details have been provided for canola seed as well as other seeds. The method is novel because no method currently exists for the determination of vigour by headspace gas analysis. The method appears to be reliable, based on the testing of more than 100 canola seed lots. The method may be suitable for a variety of crops (mostly small-seeded) based on screening tests of seed of 30 crops. One embodiment of the method uses an existing colour development technology to distinguish between high and low vigour in a simple, economical procedure suitable for an on-farm test. The colour change associated with ethanol production was demonstrated in high- and low-vigour canola seed lots.

While the preferred embodiments of the invention have been described above, it will be recognized and understood that various modifications may be made therein, and the appended claims are intended to cover all such modifications which may fall within the spirit and scope of the invention.

TABLE 1
Ethanol, acetaldehyde and unknown E (gas chromatography peak areas)
in head space gas of paired high- and low-vigour seed lots of canola.*

| | High-vigour lots | | | | Low-vigour lots | | | |
|---|---|---|---|---|---|---|---|---|
| Seed lot | Reference | Head space gasses | | | Reference | Head space gasses | | |
| pair | bioassay % | EtOH | AA | E | bioassay % | EtOH | AA | |
| 10 | 100 | 20,389 | 5,399 | 0 | 103 | 34,148 | 9,653 | |
| 12 | 100 | 5,441 | 1,872 | 0 | 89 | 16,691 | 3,390 | |
| 8 | 100 | 28,940 | 6,806 | 0 | 79 | 274,664 | 50,894 | |
| 6 | 100 | 28,391 | 8,143 | 0 | 79 | 24,240 | 4,870 | |
| 7 | 100 | 70,172 | 10,012 | 0 | 77 | 6,267,588 | 243,643 | 22, |
| 11 | 100 | 27,780 | 8,703 | 0 | 74 | 3,097,329 | 233,091 | 56, |
| 9 | 100 | 22,667 | 4,427 | 0 | 74 | 1,125,623 | 138,339 | 7, |
| 13 | 100 | 11,757 | 2,145 | 0 | 73 | 129,176 | 19,846 | |
| 2 | 100 | 300,722 | 0 | 4,591 | 71 | 6,184,369 | 89,264 | 50, |
| 5 | 100 | 38,710 | 9,463 | 0 | 67 | 4,208,946 | 262,515 | 32, |
| 3 | 100 | 75,696 | 16,472 | 0 | 65 | 6,426,057 | 333,208 | 25, |
| AC Excel | 100 | 5,724 | 2,420 | 0 | 57 | 1,439,619 | 73,715 | 19, |
| 4 | 100 | 16,354 | 3,113 | 0 | 55 | 5,030,866 | 407,121 | 78, |

*Definitions: EtOH = ethanol; AA = acetaldehyde; E = Unknown E. Reference bioassay results of low-vigour lots are expressed as percentages of paired high-vigour lots. Several varieties are represented; however, each pair consists of a single variety.

TABLE 2
Correlations between reference bioassay results
(seedling fresh weight) and quantities of low-molecular
weight volatiles found in head space gas of moist canola seed.*

Correlation with
seedling fresh weights

| Head space gasses | Correlation coefficient (r) | P** |
|---|---|---|
| Ethanol | −0.78 | <0.0001 |
| Unknown E | −0.73 | <0.0001 |
| Acetaldehyde | −0.72 | <0.0001 |
| Unknown C | −0.47 | <0.002 |
| Pentane | −0.47 | <0.0025 |
| Unknown F | −0.36 | <0.022 |
| Unknown B | −0.35 | <0.027 |
| Dimethyl sulphide | 0.18 | <0.26 |

*The sample set consisted of 93 canola seed samples and included 32 varieties or hybrids that were untreated or treated with 19 formulations of fungicide and insecticide.
**Probability of incorrectly rejecting the hypothesis that there is no relationship.

TABLE 3
Correlations between reference bioassay,
ethanol in head space gas and various vigour
test results for 17 canola seed lots.

| | Correlation coefficients (r) | |
|---|---|---|
| Vigour test | Reference bioassay | Ethanol |
| Field fresh weight at 14 d after seeding | 0.79 | −0.78 |
| Field fresh weight at 21 d after seeding | 0.80 | −0.77 |
| Field fresh weight at 28 d after seeding | 0.83 | 0.71 |
| Pre-chill test* | 0.77 | −0.77 |
| Special cold stress test** | 0.84 | −0.76 |
| Germination after 4 d on moist blotting | 0.89 | −0.81 |
| paper | | |
| Germination after 5 d on moist blotting | 0.74 | −0.79 |
| paper | | |
| Germination after 6 d on moist blotting | 0.68 | −0.72 |
| paper | | |
| Germination after 7 d on moist blotting | 0.64 | −0.68 |
| paper (standard germination test) | | |

*Seed was planted in a potting soil, chilled at 5° C. for 7 d, than warmed to 25° C. during 16-h light periods and 15° C. during 8-h dark periods for 5 d, after which the number of emerged seedlings was counted.
**Seed was planted in potting soil and sand mix (1:1), and maintained at 8° C. during 8-h light periods and 16-h dark periods for 14 d, after which the number of emerged seedlings was counted.

TABLE 4
Ethanol, acetaldehyde and Unknown E in headspace gas of barley
seed with and without a predisposition to germination loss.*

| | Germination | Ethanol | | Acetaldehyde | | Unknown E | |
|---|---|---|---|---|---|---|---|
| Lot | after aging % | Mean | SE | Mean | SE | Mean | SE |
| 1 | 22 | 1431166 | 104275 | 12947 | 1852 | 3958 | 204 |
| 2 | 45 | 424075 | 230902 | 2743 | 1152 | 1073 | 457 |
| 3 | 49 | 123547 | 103071 | 333 | 183 | 221 | 215 |
| 4 | 49 | 1530769 | 825878 | 1296 | 700 | 1169 | 566 |
| 5 | 61 | 15307 | 4295 | 220 | 105 | 41 | 41 |
| 6 | 61 | 12503 | 10431 | 181 | 116 | 0 | 0 |
| 7 | 87 | 14209 | 11668 | 125 | 44 | 47 | 47 |
| 8 | 88 | 16065 | 8917 | 303 | 109 | 43 | 43 |
| 9 | 89 | 16615 | 16615 | 9 | 9 | 71 | 71 |
| 10 | 90 | 118 | 118 | 41 | 41 | 0 | 0 |
| 11 | 91 | 253 | 253 | 65 | 33 | 0 | 0 |
| 12 | 93 | 22860 | 6336 | 680 | 275 | 30 | 30 |
| 13 | 94 | 3944 | 2055 | 163 | 47 | 32 | 32 |
| 14 | 95 | 28420 | 26712 | 146 | 102 | 37 | 37 |
| 15 | 97 | 0 | 0 | 0 | 0 | 0 | 0 |
| 16 | 98 | 7179 | 470 | 120 | 47 | 0 | 0 |
| 17 | 98 | 337929 | 326282 | 567 | 461 | 597 | 597 |
| 18 | 98 | 169 | 169 | 38 | 35 | 0 | 0 |
| 19 | 99 | 133 | 133 | 36 | 36 | 0 | 0 |
| 20 | 99 | 0 | 0 | 21 | 21 | 0 | 0 |
| 21 | 99 | 0 | 0 | 27 | 27 | 0 | 0 |
| 22 | 99 | 2037 | 1054 | 65 | 14 | 0 | 0 |
| 23 | 99 | 0 | 0 | 22 | 22 | 0 | 0 |
| 24 | 99 | 3450 | 442 | 84 | 38 | 0 | 0 |
| 25 | 99 | 1854 | 856 | 148 | 40 | 0 | 0 |
| 26 | 99 | 0 | 0 | 36 | 36 | 0 | 0 |
| 27 | 99 | 7352 | 3383 | 141 | 107 | 95 | 95 |
| 28 | 100 | 1701 | 1701 | 64 | 34 | 18 | 18 |
| 29 | 100 | 0 | 0 | 15 | 15 | 0 | 0 |
| 30 | 100 | 29 | 29 | 22 | 22 | 0 | 0 |

*Predisposition to germination loss was demonstrated by artificial aging. Prior to artificial aging, all seed lots had 95% or better germination, except for lot 4 that was 87%. Acetaldehyde, ethanol and unknown E in head space gas was determined on un-aged samples.

TABLE 5
Ethanol emissions from un-aged and artifically
aged* seed of various crops.

| | | | No. of | Ethanol (gas | | Mean ratio of |
|---|---|---|---|---|---|---|
| | | | lots/ | chromatography peak | | ethanol peak |
| Seed size | Botanical | | varieties | areas, means) | | area |
| (s, m, l) | family | Crop | tested | unaged | aged | (aged:unaged) |
| s | Brassicaceae | Canola | 5 | 507519 | 4859211 | 472 |
| s | Brassicaceae | Mustard | 2 | 1785 | 732664 | 435 |
| s | Leguminoseae | Alfalfa | 4 | 532203 | 4408895 | 352 |
| m | Cucurbitaceae | Muskmelon | 2 | 9997 | 514557 | 109 |
| s | Linaceae | Flax | 4 | 269532 | 4706536 | 83 |
| s | Brassicaceae | Turnip | 1 | 49080 | 3686979 | 75 |
| s | Brassicaceae | Radish | 1 | 7613 | 533436 | 70 |
| m | Polygonaceae | Buckwheat | 1 | 73706 | 2602294 | 35 |
| s | Brassicaceae | Cabbage | 1 | 365704 | 6992282 | 19 |
| m | Leguminoseae | Lentils | 2 | 292793 | 2742097 | 18 |
| s | Leguminoseae | Clover | 1 | 42224 | 930759 | 16 |
| l | Asteraceae | Sunflower | 1 | 91048 | 1388966 | 15 |
| m | Poaceae | Wheat (bread) | 3 | 585034 | 4238895 | 15 |
| s | Apiaceae | Carrot | 1 | 411623 | 3699238 | 9 |
| l | Leguminoseae | Soybeans | 3 | 577632 | 2173613 | 8 |
| l | Poaceae | Corn | 3 | 890299 | 5189214 | 6 |
| s | Poaceae | Ryegrass | 1 | 649565 | 4024482 | 6 |
| m | Poaceae | Oats | 4 | 858938 | 4127270 | 6 |
| m | Poaceae | Triticale | 1 | 758970 | 4113095 | 6 |
| s | Asteraceae | Lettuce | 1 | 1836862 | 10227464 | 6 |
| s | Poaceae | Canarygrass | 1 | 1302638 | 4574113 | 4 |
| l | Leguminoseae | Beans | 4 | 440974 | 1884769 | 4 |
| m | Cucurbitaceae | Watermelon | 1 | 327745 | 1359475 | 4 |
| s | Liliaceae | Onion | 1 | 1008326 | 3871595 | 4 |
| l | Leguminoseae | Peas | 4 | 1778121 | 5120460 | 4 |
| m | Poaceae | Rye | 3 | 1906312 | 5652549 | 3 |
| m | Poaceae | Wheat (durum) | 2 | 1589734 | 3974164 | 3 |
| m | Cucurbitaceae | Cucumber | 1 | 1689674 | 4134743 | 3 |
| m | Leguminoseae | Vetch | 1 | 1289992 | 2753544 | 2 |
| m | Poaceae | Barley | 5 | 2809626 | 5704220 | 2 |
| *Seed samples (4 g, except for sunflower, ryegrass and muskmelon, 2 g) were made up to 20% moisture in 10-mL head space gas analysis vials. The vials were sealed and maintained for 24 h at room temperature (un-aged samples) or at 40° C. | | | | | | |
| #(aged samples). During the period of 24-29 h after adding water, head space gas in the vials was analyzed. | | | | | | |

1. A method of measuring seed vigour comprising:
placing a seed under conditions wherein seed metabolism initiates but the seed is not germinating; and measuring the quantity of at least one gas produced by the seed.

2. The method according to claim 1 wherein the seed is canola.

3. The method according to claim 1 wherein the seed in barley.

4. The method according to claim 1 wherein the gas measured is ethanol.

5. The method according to claim 4 wherein high levels of ethanol indicate poor vigour.

6. The method according to claim 1 wherein the quantity of gas is determined by gas chromatography.

7. The method according to claim 4 wherein the quantity of ethanol is measured colourimetrically.

8. The method according to claim 4 wherein the quantity of ethanol is measured by gas detection or monitoring instrumentation.

9. The method according to claim 1 wherein the seed is a small seed.

10. The method according to claim 9 wherein the seed is selected from the group consisting of alfalfa, barley, buckwheat, cabbage, canola, clover, flax, lentils, mustard, sunflower, turnip and wheat.

11. A device for assaying seed vigour comprising:
a quantity of a detector compound that changes colour when exposed to ethanol;
an air-tight container including a sealable opening for placing seeds to be assayed within the interior of the container; and
a regulator for controlling the rate of diffusion of the ethanol from the interior of the container to the detector compound.

12. The device according to claim 11 wherein the detector compound is mounted onto an inert carrier.

13. The device according to claim 12 wherein the inert carrier is a

[symbol]

14. A kit for assaying seed vigour comprising:
a quantity of a detector compound that changes colour when exposed to ethanol;
an air-tight container including a sealable opening for placing seeds to be assayed within the interior of the container; and
a regulator for controlling the rate of diffusion of the ethanol from the interior of the container to the detector compound.
